(19) United States
(12) Patent Application Publication
(10) Pub. No.: US 2013/0060094 A1
(43) Pub. Date: Mar. 7, 2013

(54) RETRACTOR SYSTEM FOR LAPAROSCOPIC SURGERY

(71) Applicant: Jeongsam Lee, Gwangju (KR)

(72) Inventor: Jeongsam Lee, Gwangju (KR)

(21) Appl. No.: 13/695,937

(22) PCT Filed: Apr. 5, 2011
(86) PCT No.: PCT/KR11/02316

§ 371 Date: Nov. 2, 2012

(30) Foreign Application Priority Data
May 2, 2010 (KR) 10-2010-0041164
Dec. 27, 2010 (KR) 10-2010-0135709

(51) Int. Cl. A61B 1/32

(52) U.S. Cl. 600/207

(57) ABSTRACT

Disclosed is a retractor system for laparoscopic surgery. According to one aspect of the present invention, provided is a retractor system for laparoscopic surgery comprising: a sleeve portion for forming a passage at a retractor; an upper guide portion in which an upper circular elastic ring is built, formed at the upper portion of the sleeve portion; a lower guide portion comprising a circular tube in which a lower circular elastic ring is built, formed at the lower portion of the sleeve portion; a tube pressure controller for injecting air or water into the circular tube or discharging the air or water of the circular tube to contract or extend the circular tube, connected to the circular tube through a pipe; and an air injector or a CO2 injector in which one end is mounted to the adjacent area of the sleeve portion, the extended pipe passes through the inside of the lower guide portion along the outer wall of the sleeve portion, and the other end is mounted to the lower portion of the lower guide portion.

### FIELD OF THE INVENTION

The present invention relates to a retractor system for laparoscopic surgery which can guide surgical instruments inserted during the surgery to a site inside tissues to be subjected to surgery and maintain a passageway and space for easily performing the surgery.

### DESCRIPTION OF RELATED ART

Unlike existing laparotomy, laparoscopic surgery has been developed in order that an incision of skin could be incised as small as possible, and surgery utilizing the laparoscopic surgery is increased day by day. Such a laparoscopic surgery is a surgical method wherein several small holes are perforated in abdomen of a patient using trocars and surgical instrument, for example, endoscope etc., is inserted into a site in the abdominal cavity to be subjected to surgery through the trocars and thereafter surgery is performed on the site in the abdominal cavity to be subjected to surgery while viewing the site through the endoscope, and is utilized for almost all surgeries such as cholecystectomy, biliary tract stone removal, hepatolith, gastrectomy, colectomy, small bowel resection etc. Among the surgical instruments utilized for such a laparoscopic surgery, the trocar is integrally formed while an end of main body thereof communicates with a channel of fixed size, and comprises an insertion part for insertion into a hole formed in the abdomen.

In many surgical treatments, for achieving an effect of diagnosis or treatment, it is preferable to provide at least one operation channel leading to a body cavity through which various instruments pass to view, engage and treat a tissue.

In an abdominal procedure by the laparoscopic surgery, abdominal wall is pierced, and at least one tubular cannula or retractor each for forming the operation channel is inserted into the abdominal cavity (it is an empty space surrounded by peritoneum of a part of abdomen below a diaphragm, and is separated on upper side from a thorax by the diaphragm and, on lower side, adjacent to pelvis, and longitudinally surrounded by vertebra and abdominal muscle or other muscles). For viewing a site under surgery, a camera for laparoscopic surgery may be used which is connected to a monitor in surgical operating room, and can be positioned through one of the operation channels. Another instruments for laparoscopic surgery such as grasper, dissector, scissors, retractor etc. can be also positioned through at least one operation channel in order to facilitate various operations by surgeons and/or assistants for the surgeons. The retractor is a device for maintaining a wound incision and providing a passageway for operation using instruments for laparoscopic surgery. FIG. 1 is a view for explaining a structure of a conventional retractor. A retractor (200) for laparoscopic surgery comprises an upper guide (202) joined with an intermediate sleeve (203) and a lower guide (201) coupled with the intermediate sleeve (203). Commonly, the conventional retractor (200) is formed in one-piece, or is coupled with the intermediate sleeve (203) and lower guide (201) and the upper guide (202) is joined at the time of mounting. In the case of the retractor (200) as illustrated in FIG. 1, the lower guide (201) is in the form of an elastic annular ring, therefore, at the time of mounting, if the lower guide (201) is pushed through the incision of a tissue layer (210) while dented so as to inwardly concavely yield, the elastic annular ring is deployed and thus the lower guide (201) is mounted.

The conventional retractor (200) described above has to be produced in such way that the diameter of rim of the elastic annular ring is relatively large compared to the incision. If the diameter of rim of the elastic annular ring is below a certain value or the strength of material and flexibility of the elastic annular ring is weak, it is possible for the retractor (200) to depart outward from the incision during the surgery. As described above, for mounting the retractor (200) on the navel, the rim of lower housing of the retractor has to be relatively larger than the space of incision formed in the navel in order to stably mount the retractor with the tissue of abdomen interposed. In mounting or removing the retractor as described above, due to the physical shape of rim of the lower housing relatively larger than the incision, it takes a lot of time to mount and remove the retractor, and as the case may be, a problem arises that the retractor may potentially cause injury to the tissue of the patient. The wound retractor currently used for single-passageway laparoscopic surgery has problems that omentum is frequently caught between the wound retractor and front abdominal wall when the wound retractor is mounted on the navel and it is very difficult to remove the wound retractor, and surgical gloves are taken off from the wound retractor in a process of making a roof using the surgical gloves in the surgery.

Another wound retractors have too much devices mounted thereon, therefore, it takes a lot of time to mount and remove the retractors, and it costs a lot of money to produce them, thus relatively high price of product cannot be avoided. Furthermore, since a device for injection of the most of air and removal of mist generated during the surgery is mounted through trocar, problems are shown that the trocar becomes bulky and collision between the instruments frequently occurs during the surgery.

### SUMMARY OF THE PRESENT INVENTION

An object of the present invention is to provide a retractor system for laparoscopic surgery which can be easily mounted to and separated from abdominal tissue for the laparoscopic surgery. Another object of the present invention is to provide a retractor system for laparoscopic surgery which can be securely sealingly mounted to abdominal tissues of various thickness. Furthermore, yet another object of the present invention is to provide a retractor system for laparoscopic surgery wherein a soft annular ring necessary for maintaining at least basic form is housed inside a lower guide of a wound retractor and the retractor system can be easily mounted to and separated from an abdominal tissue utilizing a tubular structure which can be filled with and emptied of air or water for reinforcing the annular ring. In addition, yet another object of the present invention is to provide a retractor system for laparoscopic surgery which comprises a device for air injection passageway and passageway for removing mist generated during the surgery.

According to an aspect of the present invention, a retractor system for laparoscopic surgery is provided which comprises a sleeve portion forming a passageway in the retractor; an upper guide portion formed on a upper part of the sleeve portion and housing an upper annular elastic ring; a lower guide portion formed on a lower part of the sleeve portion and housing a lower annular elastic ring is built; and a tube pressure adjuster connected to the annular tube by a pipe and injecting air or water into the annular tube or discharging the air or water therefrom to expand or contract the annular tube.

A sheath of the upper guide portion surrounding the annular elastic ring is formed integrally with a sheath of the sleeve portion and the upper guide portion can come into close contact with a upper side of a wound incision by a process of rolling and lowering the upper annular elastic ring and the sheath of the guide portion. The tube pressure adjuster extends upward from the inside of the annular tube riding on an outer wall of the sleeve portion and is bent outward from a side surfaces of the extended sleeve portion, and a valve may be mounted at one terminal end of the tube pressure adjuster.

Furthermore, the lower annular elastic ring has low elasticity compared to the upper annular elastic ring and the upper annular elastic ring, and lower annular elastic ring and sleeve portion are flexible.

According to another aspect of the present invention, the retractor system may further comprise air injector or CO₂ injector which extends along the outer wall of the sleeve portion via the inside of the lower guide portion to a lower side of the lower guide portion.

The retractor system for laparoscopic surgery according to an example of the present invention has effects that it can be easily mounted to an abdominal tissue for the laparoscopic surgery and also can be easily separated while minimizing injury to an incision.

The retractor system for laparoscopic surgery according to an example of the present invention can be securely sealingly mounted to the abdominal tissues of various thickness so that it can be applied to any site in the laparoscopic surgery.

According to the retractor system for laparoscopic surgery according to an example of the present invention, air injection passageway and mist-removing passageway necessary for the surgery can be ensured.

The retractor system for laparoscopic surgery according to an example of the present invention can prevent omentum from being caught between the wound retractor and front abdominal wall when the retractor system for laparoscopic surgery is mounted on the navel in the laparoscopic surgery and a frame of the retractor can be maintained by injecting air or water and the wound retractor can be easily removed only by removing the air or water.

Furthermore, unlike conventional retractor, surgical gloves need not be attached to the retractor in the surgery, thus a problem can be avoided that the gloves are taken off from the wound retractor in the surgery, and cost for production of the retractor can be decreased.

Furthermore, air injection passageway and passageway for removing mist generated during the surgery are ensured, and the volume of the trocar is reduced, thus collision between laparoscopic instruments during the surgery is prevented, whereby the surgery can be smoothly performed.

### BRIEF DESCRIPTION OF THE DRAWINGS

FIG. 1 is a sectional view for explaining a structure of a conventional retractor.

FIG. 2 is a structural view of a retractor system for laparoscope according to an example of the present invention.

FIG. 3 is a sectional view of a retractor system for laparoscope according to an example of the present invention.

FIG. 4 is a sectional view of a retractor system for laparoscope wherein a lower guide portion according to an example of the present invention is expanded.

FIG. 5 is a structural view of a retractor system for laparoscope wherein a lower guide portion according to an example of the present invention is expanded.

FIG. 6 is a sectional view wherein a lower guide according to an example of the present invention is expanded and upper guide is rolled and lowered, whereby a laparoscopic system is mounted to an incision.

FIG. 7 is a sectional view illustrating the laparoscopic system comprising a protective tube according to another example of the present invention.

FIG. 8 is a sectional view wherein a laparoscopic system is mounted in which air adjuster and CO₂ tube of yet another aspect according to an example of the present invention.

FIG. 9 is a sectional view illustrating the laparoscopic system comprising a sealing cover device according to an example of the present invention.

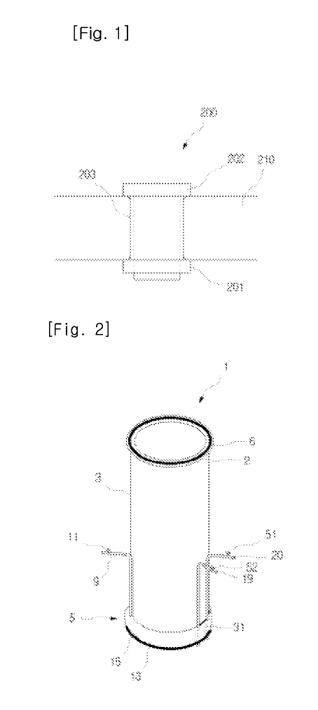

<list of reference numerals>

| | | | |
|---|---|---|---|
| 2: | upper guide portion | 3: | sleeve portion |
| 5: | lower guide portion | 6, 13: | annular elastic ring |
| 9: | tube pressure adjuster | 11: | valve |
| 19: | air injector | 20: | CO₂ injector |
| 40: | upper housing of sealing cover device | 41, 42: | trocar channel |
| 45: | coupling elastic ring | 110, 220: | tissue layer of surgical incision |

### MODES FOR CARRYING OUT THE INVENTION

A retractor system for laparoscopic surgery is disclosed.

According to an aspect of the present invention, a retractor system for laparoscopic surgery is provided which is characterized in that it comprises a sleeve portion forming a passageway in the retractor; an upper guide portion formed on a upper part of the sleeve portion and housing an upper annular elastic ring; a lower guide portion formed on a lower part of the sleeve portion and housing a lower annular elastic ring is built; a tube pressure adjuster connected to the annular tube by a pipe and injecting air or water into the annular tube or discharging the air or water therefrom to expand or contract the annular tube; and one or two air injectors or CO₂ injectors in the form of flat oval-shaped tube wherein its one terminal end is mounted around the sleeve portion and its extended pipe extends through the inside of the lower guide portion along an outer wall of the sleeve portion and thus the other terminal end is mounted to a lower part of the lower guide portion.

The present invention may be variously converted and have various examples, and specific examples are illustrated in the drawings and described in detail in a detailed description section. However, it is not intended to limit the present invention to a specific embodiment and should be understood to include all of conversions, equivalents and substitutes that are included within a conceptual and technical scope of the present invention. In description of the present invention, if detailed description of known related arts is decided to obscure the gist of the present invention, its detailed description of them is omitted.

Terms “a first˜”, “a second˜” etc. may be used for describing various components, however, the components should not be limited by such terms. The terms are used for the purpose of distinguishing a component from another components.

Terms used in the present application are used only for describing specific examples and is not intended to limit the present invention. Singular form contextually includes plural form unless specifically otherwise indicated. It should be understood that in the present application, term “comprise” or “have” etc. indicates the presence of features, numerals, steps, actions, components, parts or combination thereof mentioned in the specification and does not previously exclude a possibility of the presence or addition of one or more another features, numerals, steps, actions, components, parts or combination thereof.

Now, prescribed exemplary examples will be described for providing the overall understanding of structures, functions and principle of use of a system disclosed in the specification.

One or more embodiment of the examples is illustrated in the attached drawings.

Structures concretely described in the specification and illustrated in the attached drawings are only exemplary examples, and features illustrated or described with respect to an exemplary example may be combined with features of another examples. Such modification and alteration are intended to be included within the scope of the present invention.

Although an example of the present invention is described with respect to a laparoscopic surgery procedure in the abdominal cavity, it is to be understood that this system may be used in almost all parts of human or animal body and in procedures of surgical treatment of various another types. For example, the system disclosed as an example in the present invention can be used in thoracic cavity, pelvic cavity, cranial cavity or any natural hole of the body and also in endoscopic procedure or procedure of laparotomic surgical treatment.

Hereinafter, examples of the present invention will be described in detail with reference to the attached drawings.

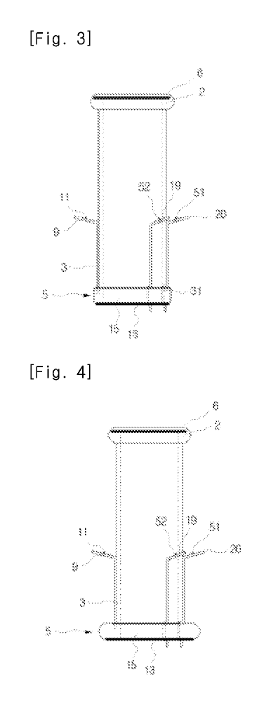

FIGS. 2 and 3 are sectional view and structural view, respectively, of a retractor system for laparoscopic surgery according to an example of the present invention.

The retractor system according to the present invention comprises a sleeve portion (3) which defines at least one operation channel extending between outer part and inner part a tissue and receives at least one laparoscopic surgery instrument for passing thereof through the operation channel.

The sleeve portion (3) according to an example of the present invention has a cylindrical structure with elasticity.

The sleeve portion (3) may be made of polymer including polycarbonate and polyetheretherketone, metal such as titanium or stainless steel, composite material such as carbon-fiber reinforced PEEK, ceramic material and any combination thereof.

Furthermore, the sleeve portion may be also made of semi-rigid/flexible material including thermoplastic elastomer such as polyurethane, polyisoprene elastomer, silicone elastomer of medium to high hardness and/or any combination thereof.

On an upper part of the sleeve portion (3), an upper guide portion (2) is formed extending coaxially with the sleeve portion (3). An upper annular elastic ring (6) may be housed inside the upper guide portion (2).

A lower guide portion (5) is formed on a lower part of the sleeve portion (3).

It is preferred that the sleeve portion (3) is made of material with a low rigidity of degree only allowing the sleeve to maintain its cylindrical form by itself and has elasticity and flexibility of degree only allowing the laparoscopic surgery instrument to be tilted when approaching at tilted angle.

Housed inside the upper guide portion (2) is an upper annular elastic ring (6) having a high elasticity and maintaining a circular form, and the upper guide portion (2) is formed with a sheath with a structure externally surrounding the annular elastic ring (6).

The sheath of the upper guide portion (2) is formed integrally with a sheath of the sleeve portion (3).

The sheath of the upper guide portion (2) may be formed of flexible elastomer such as rubber, urethane, vinyl and the like. If the sheath of the upper guide portion (2) housing the annular elastic ring is rolled to be lowered, accordingly the sheath of the sleeve is rolled to be lowered, and as a result, the height of a lower part of the rolled and lowered sleeve portion (3) of the retractor system becomes equal to that of the incision surface of the tissue layer (201). Namely, the height of the retractor system inserted in the incision can be adjusted by rolling and lowering the sheath of the upper guide portion (2).

For the sleeve portion (3) according to an example of the present invention, its sheath and main body (30) are integrally formed of flexible elastomer such as rubber, urethane, vinyl and the like, or the main body (30) may be formed of silicone elastomer of high elasticity and high hardness while the sheath may be formed of flexible elastomer such as rubber, urethane, vinyl and the like.

The inner diameter of the lower guide portion (5) is equal to the diameter of the sleeve portion (3) and the lower guide portion is kept coaxial with the sleeve and formed in the form of annular tube (15) whose outer diameter is larger than the diameter of the sleeve portion (3) by 20%˜30% or more.

The lower guide portion (5) houses thereinside an annular elastic ring (13) having a lower elasticity than the elastic ring (6) of the upper guide portion, and tube pressure adjusters (9) for injecting air into the lower guide portion may be mounted riding on a wall of the sleeve portion (3).

Furthermore, the tube pressure adjuster (9) extends upward from the inside of the annular tube (15) of lower guide portion (5) over a certain length riding on the outer wall of the sleeve portion (3) and is bent outward from circumferential side surface of the sleeve portion (3).

Air valve (11) and injection port are mounted at a terminal end of the tube pressure adjusters (9).

The lower guide portion (5) and upper guide portion (2) are formed of semi-rigid/flexible material including polymer including polycarbonate and polyetheretherketone, thermolplastic elastomer such as polyurethane, polyisoprene elastomer, silicone elastomer of medium to high hardness, rubber and any combination thereof, and thus can be elastically stretched upon injection of air or water.

The upper and lower guide portions and sleeve portion may be formed in one-piece or from at least two separate components of composite material conforming to each other.

The diameter of the sleeve portion according to an example of the present invention is about 20˜100 mm for enabling the sleeve portion to be applied for holding an incision lance through the navel (the actual diameter of the incision lance is 10˜25 mm, but a space is enlarged to the order of 20˜50 mm if the sleeve portion is mounted), and the length of the sleeve portion is 100˜200 mm including the depth of the incision for surgery and the length over which the upper guide portion is rolled. Namely, the diameter and length of the sleeve portion according to an example of the present invention may be variously determined depending on sites to be subjected to surgery.

Furthermore, for the laparoscopic surgery retractor system according to an example of the present invention, air adjuster (19) for adjustment of mist in the abdominal cavity and CO₂ injector (20) may be installed so as lead to the inside of abdominal cavity via the sleeve portion (3).

The CO₂ injector (20) is used for injecting CO₂ gas in an abdominal procedure by means of laparoscopic surgery. For example, CO₂ gas of about 10˜15 mmHg is injected into the abdominal cavity in order to maintain positive pressure therein.

Furthermore, the air injector (19) can remove the mist out of the abdominal cavity by connecting a mist-removing device or repeatedly opening and closing a valve during the surgery for removing the mist generated during the surgery.

As illustrated in FIGS. 2 and 3, the air injector (19) and CO₂ injector (20) may extend from the middle of the sleeve portion (3) along the outer wall of the sleeve portion (3) via the inside of the lower guide portion (5) to a lower side of the lower guide portion (5).

Furthermore, at the lower terminal end of the air injector (19) or CO₂ injector (20), a tube guide portion (31) may be further formed which surrounds a tube of expanded shape of the lower guide portion (5). By means of the tube guide portion (31), the lower guide portion (5) can maintain its expanded thickness constantly when the lower guide portion is expanded by the injected air.

Furthermore, the tube of the air injector (19) or CO₂ injector (20) may be formed in the form of flat oval in order to maintain sealing with the incision and not to interfere the expanding of the annular tube (15).

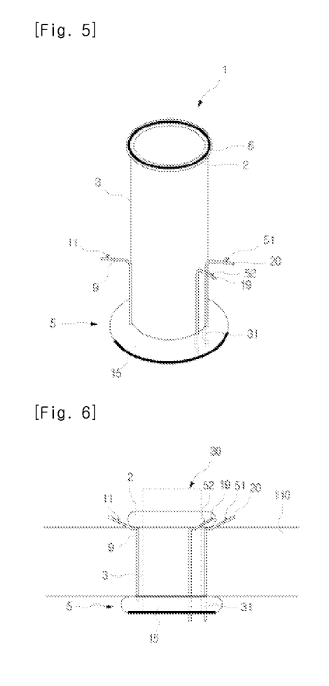

FIGS. 4 and 5 are views illustrating a situation where air is injected in the annular tube of the lower guide portion, and FIG. 6 is a sectional view illustrating a situation where the laparoscopic surgery retractor system according to an example of the present invention is mounted to the surgical incision.

The following is description of an operation method using the laparoscopic surgery retractor system (1) according to an example of the present invention.

It is the navel that is used for securing a surgical incision window in the single-passageway laparoscopic surgery, wherein the navel is incised upward and downward therefrom in each case over a length of 10 to 25 mm, thereby forming the surgical incision and thereafter the annular elastic ring (13) housed in the lower guide portion of the retractor system (1) for laparoscope according to an example of the present invention is rolled and then pushed into the inside of abdomen through the surgical incision of the navel. At this time, the annular tube (15) is pushed while contracted due to removal of air.

Since the lower annular elastic ring (13) has a soft structure of low elasticity compared to the upper annular elastic ring (6), the lower annular elastic ring can be easily pushed into the incision opening relatively smaller than the lower annular elastic ring (13) in a situation where air has been removed.

Next, if air is injected into the annular tube (15) of the lower guide portion (5) by means of syringe(not shown) and the like coupled to an injection port of the tube pressure adjuster (9) and then the valve is closed, the lower guide portion (5) according to an example of the present invention is expanded to be fixedly retained on a lower side of the incision of the abdominal cavity, as illustrated in FIGS. 4 and 5.

Thereafter, if the sheath surrounding the upper annular elastic ring (6) of the upper guide portion is rolled and lowered along the sleeve portion (3) together with the sheath of the sleeve portion (3) to an upper side of a tissue layer (110) of the incision as illustrated in FIG. 6, an upper side of the tissue layer (110) and the sheath of the upper guide portion (2) surrounding the upper annular elastic ring come into close contact with each other. Therefore, the tissue layer (110) of the surgical incision can be sealed

on upper and lower sides by the upper guide portion (2) and the expanded lower guide portion (5) with the sleeve portion (3) interposed therebetween, thus a passageway for surgery is ensured for the inside and outside of the abdominal cavity.

Thereafter. as shown in FIG. 8, if an upper housing (40) of cover device for sealing the upper guide portion (2) is mounted, both spaces inside and outside the abdominal cavity can be isolated from each other.

Thereafter, by opening the valve of the CO₂ injector (20), CO₂ gas is injected into the abdominal cavity, thereby maintaining positive pressure therein, whereby necessary space field of vision can be ensured.

Furthermore, the mist can removed out of the abdominal cavity by connecting the mist-removing device to the air injector (19) or repeatedly opening and closing the valve of the air injector (19) during the surgery for removing the mist generated during the surgery.

Next, after the surgery has been completed, if air in the annular tube (15) of the lower guide portion (5) is removed by opening the valve (11) of the tube pressure adjuster (9) or drawing the air by means of the syringe and the like, force restraining the sleeve portion (3) in upward and downward directions on the lower side of the tissue layer (110) is weaken, thus the retractor system (1) can be easily separated and removed from the incision only by action of lifting the upper guide portion.

In the example of the present invention described above, the annular tube (15) of the lower guide portion (5) is expanded by injection of air, however, in another example of the present invention, for substance injected into the tube pressure adjuster (9), air may be substituted by water.

Namely, by injecting the water into the annular tube (15) by means of the tube pressure adjuster, the lower guide portion (5) is expanded and then the retractor system is mounted, and thereafter when the retractor system is to be removed, the water in the annular tube is discharged by operating the valve (11) or is suck out by means of the syringe and the like after opening the valve (11), whereby the retractor system can be easily removed from the incision without causing damage to the wound.

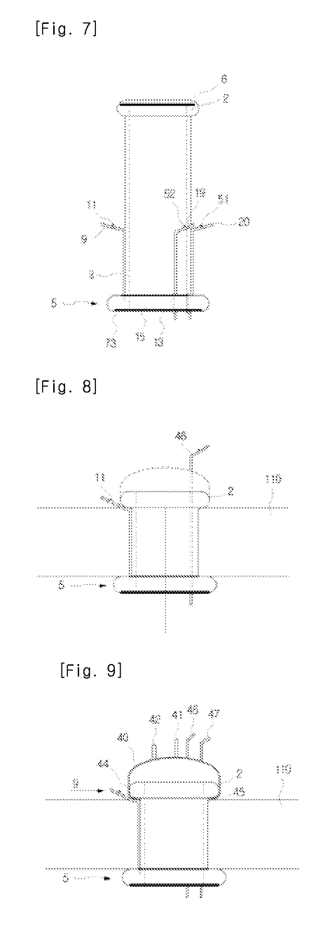

FIG. 7 is a sectional view illustrating a situation where a retractor system according to another example of the present invention is mounted.

The annular tube described above has a concern that the tube may be broken by contacting with surgical instruments around the wound while expanded by air or water. For reinforcing the annular tube, in one example, a protective tube (73) surrounding the outside of the annular tube (15) is further included.

A spacer rib (not illustrated) made of resilient material such as urethane and the like may be mounted on the protective tube (73) for maintaining a constant spacing between the protective tube and the annular tube.

Even if the lower guide portion (5) is partially damaged by the surgical instruments, the protective tube (73) only is partially broken and the expanded annular tube (15) can securely maintain its mounted state while expanded.

FIG. 8 is a sectional view illustrating a situation where a laparoscopic system of yet another aspect according to an example of the present invention is mounted.

In the example of FIG. 8, tubes of the air injector (19) and CO₂ injector (20) extend through the inside of the sleeve portion (3) to an upper end of the upper guide portion (2).

As illustrated in FIG. 8, if the tubes of the air injector (19) and CO₂ injector (20) extend to the upper end of the upper guide portion (2), the tubes can be connected to upper channels (46, 47) of the upper housing (40) of the sealing cover device (40) of FIG. 9 described later. Accordingly, sealing structure can be well maintained, compared to a case where tube paths are formed between the incision (110) and sleeve portion (3).

After the laparoscopic surgery retractor system of the present invention has been mounted, a separate sealing cover device may be mounted over the upper guide portion (2).

FIG. 9 is a sectional view illustrating a situation where a sealing cover device suitable for the surgical retractor of the present invention is mounted.

The sealing cover device comprises a lower coupling part (44) housing an coupling elastic ring (45) at a terminal end and an upper housing (40) where trocar channels (41, 42) are formed into and from which the laparoscopic surgery instruments can be introduced and removed.

The coupling elastic ring (45) housed in the lower coupling part (44) is formed with a diameter slightly less than the outer diameter of the upper guide portion (2), and if the coupling elastic ring (45) is stretched to cover the upper guide portion (2), the sealing cover device can be easily mounted on the retractor system.

If the upper housing (40) of the sealing cover device is mounted on the upper guide portion (2), both spaces inside and outside the abdominal cavity can be isolated from each other.

The retractor system for laparoscopic surgery of the present invention is applicable to industry of medical instrument related to laparoscopic or thoracoscpic surgery or endoscopic surgery of various types, particularly, a single-passageway laparoscopic surgery.

What is claimed:

1. A retractor system for laparoscopic surgery comprising:
a sleeve portion forming a passageway in the retractor;
an upper guide portion formed on a upper part of the sleeve portion and housing an upper annular elastic ring;
a lower guide portion formed on a lower part of the sleeve portion and housing a lower annular elastic ring is built;
a tube pressure adjuster connected to the annular tube by a pipe and injecting air or water into the annular tube or discharging the air or water therefrom to expand or contract the annular tube; and
an air injector or CO₂ injector wherein its one terminal end is mounted around the sleeve portion and its extended pipe extends through the inside of the lower guide portion along an outer wall of the sleeve portion and thus the other terminal end is mounted to a lower part of the lower guide portion.

2. The retractor system for laparoscopic surgery according to claim 1, wherein a sheath of the upper guide portion surrounding the annular elastic ring is formed integrally with a sheath of the sleeve portion and the upper guide portion comes into close contact with a peripheral upper side of a wound incision by a process of rolling and lowering the upper annular elastic ring and the sheath of the guide portion.

3. The retractor system for laparoscopic surgery according to claim 2, wherein the sheath of the sleeve portion and its inner main body are integrally formed of flexible elastomer or the inner main body is formed of silicone elastomer of high hardness while the sheath of the sleeve portion is formed of flexible elastomer.

4. The retractor system for laparoscopic surgery according to claim 1, wherein one terminal end of the tube pressure adjuster is mounted inside the annular tube and an extended tube of the adjuster extends upward over a certain length riding on an outer wall of the sleeve portion and is bent outward from circumference of the sleeve portion, and a valve and an injection port are mounted at the other terminal end of the tube pressure adjuster.

5. The retractor system for laparoscopic surgery according to claim 1, wherein the upper annular elastic ring and lower annular elastic ring are flexible, and the lower annular elastic ring is made of soft material and has low elasticity compared to the upper annular elastic ring.

6. The retractor system for laparoscopic surgery according to claim 1, wherein the sleeve portion is flexible.

7. The retractor system for laparoscopic surgery according to claim 1, wherein after the lower guide portion of the retractor system is rolled and inserted toward a lower side of surgical incision, air or water is injected into the annular tube of the lower guide portion through the injection port of the tube pressure adjuster to maintain the annular tube in its expanded state, and the sheath of the upper guide portion surrounding the upper annular elastic ring of the upper guide portion is rolled and lowered together with the sheath of the sleeve portion to a peripheral upper side of the surgical incision, whereby the retractor system is mounted so that a tissue layer of the surgical incision can be sealed on upper and lower sides by the upper guide portion and lower guide portion with the sleeve portion interposed therebetween.

8. The retractor system for laparoscopic surgery according to claim 1, wherein the lower guide portion further comprises a protective tube surrounding the outside of the annular tube.
